(19) United States
(12) Patent Application Publication
(10) Pub. No.: US 2013/0344087 A1
(43) Pub. Date: Dec. 26, 2013

(54) COMBINATIONS OF ANTI-S1P ANTIBODIES AND SHPINGOLIPID PATHWAY INHIBITORS

(71) Applicants: Roger A. Sabbadini, Lakeside, CA (US); Jonathan Michael Wojciak, Encinitas, CA (US)

(72) Inventors: Roger A. Sabbadini, Lakeside, CA (US); Jonathan Michael Wojciak, Encinitas, CA (US)

(21) Appl. No.: 13/702,551

(22) PCT Filed: Jun. 4, 2011
(86) PCT No.: PCT/US11/39200

§ 371 Date: Sep. 9, 2013

Related U.S. Application Data
(60) Provisional application No. 61/351,904, filed on Jun. 6, 2010.

(51) Int. Cl. C07K 16/18

(52) U.S. Cl. CPC C07K 16/18
USPC 424/172.1; 530/389.8; 530/387.3; 530/388.2

(57) ABSTRACT

Methods for administering combinations of compositions comprising anti-S1P antibodies or antibody fragments and modulators of sphingolipid metabolic pathway enzymes are described. Such methods allow aberrant or undesirable levels of S1P to be reduced in patients known or suspected to have a disease or disorder correlated with aberrant S1P levels, and thus will be useful in treating such diseases and disorders.

### 1. FIELD OF THE INVENTION

The present invention relates to methods of using antibodies reactive sphingosine-1-phosphate (S1P) in combination with sphingolipid metabolic pathway inhibitors to reduce the effective concentration of S1P. Such methods can be used to treat diseases and disorders correlated with aberrant or otherwise undesired amounts or activity of S1P.

### 2. BACKGROUND THE INVENTION

The following description includes information that may be useful in understanding the present invention. It is not an admission that any of the information provided herein, or any publication specifically or implicitly referenced herein, is prior art, or even particularly relevant, to the presently claimed invention.

Bioactive Signaling Lipids

Lipids and their derivatives are now recognized as important targets for medical research, not as just simple structural elements in cell membranes or as a source of energy for β-oxidation, glycolysis or other metabolic processes. In particular, certain bioactive lipids function as extracellular and/or intracellular signaling mediators important in animal and human disease. “Lipid signaling” refers to any of a number of cellular signal transduction pathways that use cell membrane lipids as second messengers, as well as referring to direct interaction of a lipid signaling molecule with its own specific receptor. Lipid signaling pathways are activated by a variety of extracellular stimuli, ranging from growth factors to inflammatory cytokines, and regulate cell fate decisions such as apoptosis, differentiation, and proliferation. Research into bioactive lipid signaling is an area of intense scientific investigation as more and more bioactive lipids are identified and their actions characterized.

Examples of bioactive lipids include the sphingolipids, which include sphingomyelin, ceramide, ceramide-1-phosphate, sphingosine, sphingosylphosphoryl choline, sphinganine, sphinganine-1-phosphate (Dihydro-S1P) and sphingosine-1-phosphate. Sphingolipids and their derivatives represent a group of extracellular and intracellular signaling molecules with pleiotropic effects on important cellular processes. Other examples of bioactive signaling lipids include phosphatidylserine (PS), phosphatidylinositol (PI), phosphatidylethanolamine (PEA), diacylglyceride (DG), sulfatides, gangliosides, cerebrosides, the eicosanoids (including the cannabinoids, leukotrienes, prostaglandins, lipoxins, epoxyeicosatrienoic acids, and isoeicosanoids) such as the hydroxyeicosatetraenoic acids (HETEs, including 5-HETE, 12-HETE, 15-HETE and 20-HETE), non-eicosanoid cannabinoid mediators, phospholipids and their derivatives such as phosphatidic acid (PA) and phosphatidylglycerol (PG), platelet activating factor (PAF) and cardiolipins as well as lysophospholipids such as lysophosphatidyl choline (LPC) and various lysophosphatidic acids (LPA).

Sphingolipids are a unique class of lipids that were named, due to their initially mysterious nature, after the Sphinx. Sphingolipids were initially characterized as primary structural components of cell membranes, but recent studies indicate that sphingolipids also serve as cellular signaling and regulatory molecules. The sphingolipid signaling mediators ceramide (CER), sphingosine (SPH), and sphingosine-1-phosphate (SIP) have been most widely studied and have recently been appreciated for their roles in the cardiovascular system, angiogenesis, and tumor biology. For a review of sphingolipid metabolism, see Liu, et al., Crit. Rev. Clin. Lab. Sci. 36:511-573, 1999. For reviews of the sphingomyelin signaling pathway, see Hannun, et al., Adv. Lipid Res. 25:27-41, 1993; Liu, et al., Crit. Rev. Clin. Lab. Sci. 36:511-573, 1999; Igarashi, J. Biochem. 122:1080-1087, 1997; Oral, et al., J. Biol. Chem. 272:4836-4842, 1997; and Spiegel et al., Biochemistry (Moscow) 63:69-83, 1998.

S1P is a mediator of cell proliferation and protects from apoptosis through the activation of survival pathways. It has been suggested that the balance between CER/SPH levels and S1P provides a rheostat mechanism that decides whether a cell is directed into the death pathway or is protected from apoptosis. The key regulatory enzyme of the rheostat mechanism is sphingosine kinase (SPHK) whose role is to convert the death-promoting bioactive signaling lipids (CER/SPH) into the growth-promoting S1P. S1P has two fates: S1P can be degraded by S1P lyase, an enzyme that cleaves S1P to phosphoethanolamine and hexadecanal, or, less common, hydrolyzed by S1P phosphatase to SPH.

The pleiotropic biological activities of S1P are mediated via a family of G protein-coupled receptors (GPCRs) originally known as Endothelial Differentiation Genes (EDG). Five GPCRs have been identified as high-affinity S1P receptors (S1PRs): S1P₁/EDG-1, S1P₂/EDG-5, S1P₃/EDG-3, S1P₄/EDG-6, and S1P₅/EDG-8 only identified as late as 1998. Many responses evoked by S1P are coupled to different heterotrimeric G proteins (G_{q-}, Gᵢ, G₁₂₋₁₃) and the small GTPases of the Rho family.

In adults, S1P is released from platelets and mast cells to create a local pulse of free S1P (sufficient enough to exceed the K_{d} of the S1PRs) for promoting wound healing and participating in the inflammatory response. Under normal conditions, the total S1P in the plasma is quite high (300-500 nM), although most S1P may be ‘buffered’ by serum proteins, particularly lipoproteins (e.g., HDL>LDL>VLDL) and albumin, so that the bio-available S1P (or the free fraction of S1P) is insufficient to appreciably activate S1PRs. If this were not the case, inappropriate angiogenesis and inflammation could result. Intracellular actions of S1P have also been suggested.

Widespread expression of the cell surface S1P receptors allows S1P to influence a diverse spectrum of cellular responses, including proliferation, adhesion, contraction, motility, morphogenesis, differentiation, and survival. This spectrum of response appears to depend upon the overlapping or distinct expression patterns of the S1P receptors within the cell and tissue systems. In addition, crosstalk between S1P and growth factor signaling pathways, including platelet-derived growth factor (PDGF), vascular endothelial growth factor (VEGF), and basic fibroblastic growth factor (bFGF), have recently been reported. The regulation of various cellular processes involving S1P has particular impact on neuronal signaling, vascular tone, wound healing, immune cell trafficking, reproduction, and cardiovascular function, among others. Alterations of endogenous levels of S1P within these systems can have detrimental effects, eliciting several pathophysiological conditions, including cancer, inflammation, angiogenesis, heart disease, asthma, and autoimmune diseases.

Until recently, sphingolipid-based treatment strategies focused on targeting key enzymes of the sphingolipid metabolic pathway, such as SPHK. See FIG. 1. More recently, Sabbadini and colleagues have developed a novel approach to the treatment of various S1P-correlated diseases and disorders, including cardiovascular diseases, cerebrovascular diseases, ocular disease, and various cancers, that involves reducing levels of biologically available S1P using antibodies specific for S1P, either alone or in combination with other treatments. Interference with the lipid mediator S1P was not previously emphasized, largely because of difficulties in directly mitigating this lipid target, in particular because of the difficulty first in raising and then in detecting antibodies against S1P. Recently, however, the successful generation of antibodies specific for S1P has been described. See, e.g., commonly owned, U.S. patent application Ser. No. 11/588,973 and published PCT application WO2007/053447. Such antibodies, which can, for example, selectively adsorb S1P from serum, act as molecular sponges to neutralize extracellular S1P. See also commonly owned U.S. Pat. Nos. 6,881,546 and 6,858,383 and U.S. patent application Ser. No. 10/029,372. SPHINGOMAB™, the murine monoclonal antibody (mAb) developed by Lpath, Inc. and described in certain patents or patent applications listed above, has been shown to be effective in models of human disease. In some situations, a humanized antibody may be preferable to a murine antibody, particularly for therapeutic uses in humans, where human-anti-mouse antibody (HAMA) response may occur. Such a response may reduce the effectiveness of the antibody by neutralizing the binding activity and/or by rapidly clearing the antibody from circulation in the body. The HAMA response can also cause toxicities with subsequent administrations of mouse antibodies.

A first-in-class humanized anti-S1P antibody (Sonepcizumab, LT1009) has now been developed. See, e.g., commonly owned U.S. patent application Ser. Nos. 11/924,890,12/258,337, 12/258,346, 12/258,353, 12/258,355, 12/258,383, 12/690,033, and 12/794,668. This antibody, as well as its derivatives and variants, has the advantages of the murine mAb in terms of efficacy in binding S1P, neutralizing S1P, and modulating disease states related to S1P, but lacks the potential disadvantages of the murine mAb when used in a human context. Indeed, the humanized LT1009 antibody has activity greater than that of the parent (murine) antibody in animal models of disease and is currently undergoing clinical trials for cancer and age-related macular degeneration.

In the course of conducting the foregoing human clinical studies of Sonepcizumab (LT1009) it was discovered that the absolute concentration of S1P increased in a does-dependent manner, although the amount of bioavailable S1P did not.

### 3. DEFINITIONS

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) of the parent antibody, or variants thereof that retain antigen binding activity. Antibodies are defined herein as retaining at least one desired activity of the parent antibody. Desired activities can include the ability to bind the antigen specifically, the ability to inhibit proliferation in vitro, the ability to inhibit angiogenesis in vivo, and the ability to alter cytokine profile(s) in vitro.

An “antibody derivative” is an immune-derived moiety, i.e., a molecule that is derived from an antibody. This includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)₂, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

An “antibody variant” refers herein to a molecule which differs in amino acid sequence from the amino acid sequence of a native or parent antibody that is directed to the same antigen by virtue of addition, deletion, and/or substitution of one or more amino acid residue(s) in the antibody sequence and which retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable region or a constant region of a light chain and/or a heavy chain, including in the Fc region, the Fab region, the CH1 domain, the CH2 domain, the CH3 domain, and the hinge region.

An “anti-S1P agent” refers to any therapeutic agent that binds S1P, and includes antibodies, antibody variants, antibody-derived molecules or non-antibody-derived moieties that bind LPA and its variants.

An “anti-S1P antibody” or an “immune-derived moiety reactive against S1P” refers to any antibody or antibody-derived molecule that binds S1P. As will be understood from these definitions, antibodies or immune-derived moieties may be polyclonal or monoclonal and may be generated through a variety of means, and/or may be isolated from an animal, including a human subject.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or GPCRs or enzymes or factors that, in turn, activate complex signaling systems that result in changes in cell function or survival. As intracellular mediators, bioactive lipids can exert their actions by directly interacting with intracellular components such as enzymes, ion channels or structural elements such as actin. Specifically excluded from the class of bioactive lipids according to the invention are phosphatidylcholine and phosphatidylserine, as well as their metabolites and derivatives that function primarily as structural members of the inner and/or outer leaflet of cellular membranes.

The term “biologically active,” in the context of an antibody or antibody fragment or variant, refers to an antibody or antibody fragment or antibody variant that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body which has a particular molecular feature that makes it useful for measuring the progress of disease or the effects of treatment. For example, S1P is a biomarker for certain hyperproliferative and/or cardiovascular conditions.

The term “cardiotherapeutic agent” refers to an agent that is therapeutic to diseases and diseases caused by or associated with cardiac and myocardial diseases and disorders.

“Cardiovascular therapy” encompasses cardiac therapy (treatment of myocardial ischemia and/or heart failure) as well as the prevention and/or treatment of other diseases associated with the cardiovascular system, such as heart disease. The term “heart disease” encompasses any type of disease, disorder, trauma or surgical treatment that involves the heart or myocardial tissue. Of particular interest are conditions associated with tissue remodeling. The term “cardiotherapeutic agent” refers to an agent that is therapeutic to diseases and diseases caused by or associated with cardiac and myocardial diseases and disorders.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of carriers suitable for use in accordance with the invention are known in the art. These, as well as later discovered or invented naturally occurring or synthetic carriers, can be adapted for application in accordance with the invention.

“Cerebrovascular therapy” refers to therapy directed to the prevention and/or treatment of diseases and disorders associated with cerebral ischemia and/or hypoxia. Of particular interest are cerebral ischemia and/or hypoxia resulting from global ischemia resulting from a heart disease, including without limitation heart failure.

The term “chemotherapeutic agent” means anti-cancer and other anti-hyperproliferative agents. Thus chemotherapeutic agents are a subset of therapeutic agents in general. Chemotherapeutic agents include, but are not limited to: DNA damaging agents and agents that inhibit DNA synthesis: anthracyclines (doxorubicin, donorubicin, epirubicin), alkylating agents (bendamustine, busulfan, carboplatin, carmustine, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine), platinum derivatives (cisplatin, carboplatin, cis diammine-dichloroplatinum), and topoisomerase inhibitors (Camptosar); anti-metabolites such as capecitabine, chlorodeoxyadenosine, cytarabine (and its activated form, ara-CMP), cytosine arabinoside, dacabazine, floxuridine, fludarabine, 5-fluorouracil, 5-DFUR, gemcitabine, hydroxyurea, 6-mercaptopurine, methotrexate, pentostatin, trimetrexate, 6-thioguanine); anti-angiogenics (bevacizumab, thalidomide, sunitinib, lenalidomide, TNP-470, 2-methoxyestradiol, ranibizumab, sorafenib, erlotinib, bortezomib, pegaptanib, endostatin); vascular disrupting agents (flavonoids/flavones, DMXAA, combretastatin derivatives such as CA4DP, ZD6126, AVE8062A, etc.); biologics such as antibodies (Herceptin, Avastin, Panorex, Rituxin, Zevalin, Mylotarg, Campath, Bexxar, Erbitux); endocrine therapy: aromatase inhibitors (4-hydroandrostendione, exemestane, aminoglutehimide, anastrazole, letozole), anti-estrogens (Tamoxifen, Toremifine, Raoxifene, Faslodex), steroids such as dexamethasone; immuno-modulators: cytokines such as IFN-beta and IL2), inhibitors to integrins, other adhesion proteins and matrix metalloproteinases); histone deacetylase inhibitors like suberoylanilide hydroxamic acid; inhibitors of signal transduction such as inhibitors of tyrosine kinases like imatinib (Gleevec); inhibitors of heat shock proteins like 17-N-allylamino-17-demethoxygeldanamycin; retinoids such as all trans retinoic acid; inhibitors of growth factor receptors or the growth factors themselves; anti-mitotic compounds and/or tubulin-depolymerizing agents such as the taxoids (paclitaxel, docetaxel, taxotere, BAY 59-8862), navelbine, vinblastine, vincristine, vindesine and vinorelbine; anti-inflammatories such as COX inhibitors and cell cycle regulators, e.g., check point regulators and telomerase inhibitors.

The term “chimeric” antibody (or immunoglobulin) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., infra; Morrison et al., Proc. Natl. Acad. Sci. U.S.A., vol. 81:6851 (1984)).

The term “combination therapy” generally refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and an anti-S1P antibody, or two different antibodies. In the context of this invention, a combination therapy comprises administration of an anti-S1P antibody and a second chemically distinct active ingredient directed at modulating sphingolipid metabolism, for example, by inhibiting an enzyme such as SPHK. The methods of the invention may also further comprise the delivery of another treatment, such as radiation therapy and/or surgery and/or administration of one or more other biological agents (e.g., anti-VEGF, TGFβ, PDGF, or bFGF agent), chemotherapeutic agents, or other drugs. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, drug-based portions of a combination therapy may be delivered before or after surgery or radiation treatment.

“Effective concentration” refers to the absolute, relative, and/or available concentration and/or activity, for example, of certain undesired bioactive lipids. In other words, the effective concentration of a bioactive lipid is the amount of lipid available, and able, to perform its biological function. In the present invention, an immune-derived moiety such as, for example, a monoclonal antibody directed to S1P is able to reduce the effective concentration of S1P by binding to the lipid and rendering it unable to perform its biological function. In this example, the lipid itself is still present (it is not degraded by the antibody, in other words) but can no longer available to be bound by its receptor or other targets to cause a downstream effect. As will be appreciated, “effective concentration” as well as absolute concentration of S1P in a biological sample (e.g., whole blood or blood serum or plasma) can be determined using any suitable method or assay now know or later developed for directly and/or indirectly measuring the effective and/or absolute concentration of S1P in a patient, or in a biological sample taken from a patient.

A “fully human antibody” can refer to an antibody produced in a genetically engineered (i.e., transgenic) mouse (e.g. from Medarex) that, when presented with an immunogen, can produce a human antibody that does not necessarily require CDR grafting. These antibodies are fully human (100% human protein sequences) from animals such as mice in which the non-human antibody genes are suppressed and replaced with human antibody gene expression. The applicants believe that antibodies could be generated against bioactive lipids when presented to these genetically engineered mice or other animals who might be able to produce human frameworks for the relevant CDRs.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding non-human residues.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones et al., Nature 321:522-525 (1986); Reichmann et al., Nature 332:323-329 (1988); and Presta, Curr. Op. Struct. Biol. 2:593-596 (1992) and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include but are not limited to disorders of excessive scarring (i.e., fibrosis) such as age-related macular degeneration, cardiac remodeling and failure associated with myocardial infarction, excessive wound healing such as commonly occurs as a consequence of surgery or injury, keloids, and fibroid tumors and stenting.

An “immune-derived moiety” includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope (see, e.g., Immunobiology, 5th Edition, Janeway, Travers, Walport, Shlomchiked. (editors), Garland Publishing (2001)). In the present invention, the antigen is a lipid molecule, such as a bioactive lipid molecule.

To “inhibit,” particularly in the context of a biological phenomenon, means to decrease, suppress, or delay. For example, a treatment yielding “inhibition of tumorigenesis” may mean that tumors do not form at all, or that they form more slowly, or are fewer in number than in the untreated control.

In the context of this invention, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The term “monoclonal antibody” (mAb) as used herein refers to an antibody obtained from a population of substantially homogeneous antibodies, or to said population of antibodies. The individual antibodies comprising the population are essentially identical, except for possible naturally occurring differences that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Monotherapy” refers to a treatment regimen based on the delivery of one therapeutically effective compound, whether administered as a single dose or several doses over time.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

The “parent” antibody herein is one that is encoded by an amino acid sequence used for the preparation of the variant. The parent antibody may be a native antibody or may already be a variant, e.g., a chimeric antibody. For example, the parent antibody may be a humanized or human antibody.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds of this invention and which are is biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J. Pharm. Sci., vol. 66, 1-19).

A “plurality” means more than one.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

The term “species” is used herein in various contexts, e.g., a particular species of chemotherapeutic agent. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus an antibody is commonly said to “bind” (or “specifically bind”) or be “reactive with” (or “specifically reactive with), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds S1P”, an “antibody reactive against S1P”, an “antibody reactive with S1P”, an “antibody to S1P”, and an “anti-S1P antibody” have the same meaning. Antibody molecules can be tested for specificity of binding by comparing binding to the desired antigen to binding to unrelated antigen or analogue antigen or antigen mixture under a given set of conditions. Preferably, an antibody according to the invention will lack significant binding to unrelated antigens, or even analogs of the target antigen. “Specifically associate” and “specific association” and the like refer to a specific, non-random interaction between two molecules, which interaction depends on the presence of structural, hydrophobic/hydrophilic, and/or electrostatic features that allow appropriate chemical or molecular interactions between the molecules.

The term “sphingolipid” as used herein refers to the class of compounds in the art known as sphingolipids, including, but not limited to the following compounds (see http//www.lipidmaps.org for chemical formulas, structural information, etc. for the corresponding compounds):

Sphingoid bases [SP01]

Sphing-4-enines (Sphingosines) [SP0101]

Sphinganines [SP0102]

4-Hydroxysphinganines (Phytosphingosines) [SP0103]

Sphingoid base homologs and variants [SP0104]

Sphingoid base 1-phosphates [SP0105]

Lysosphingomyelins and lysoglycosphingolipids [SP0106]

N-methylated sphingoid bases [SP0107]

Sphingoid base analogs [SP0108]

Ceramides [SP02]

N-acylsphingosines (ceramides) [SP0201]

N-acylsphinganines (dihydroceramides) [SP0202]

N-acyl-4-hydroxysphinganines (phytoceramides) [SP0203]

Acylceramides [SP0204]

Ceramide 1-phosphates [SP0205]

Phosphosphingolipids [SP03]

Ceramide phosphocholines (sphingomyelins) [SP0301]

Ceramide phosphoethanolamines [SP0302]

Ceramide phosphoinositols [SP0303]

Phosphonosphingolipids [SP04]

Neutral glycosphingolipids [SP05]

Simple Glc series (GlcCer, LacCer, etc) [SP0501]

GalNAcb1-3Gala1-4Galb1-4Glc-(Globo series) [SP0502]

GalNAcb1-4Galb1-4Glc-(Ganglio series) [SP0503]

Galb1-3GlcNAcb1-3Galb1-4Glc-(Lacto series) [SP0504]

Galb1-4GlcNAcb1-3Galb1-4Glc-(Neolacto series) [SP0505]

GalNAcb1-3Gala1-3Galb1-4Glc-(Isoglobo series) [SP0506]

GlcNAcb1-2Mana1-3Manb1-4Glc-(Mollu series) [SP0507]

GalNAcb1-4GlcNAcb1-3Manb1-4Glc-(Arthro series) [SP0508]

Gal-(Gala series) [SP0509]

Other [SP0510]

Acidic glycosphingolipids [SP06]

Gangliosides [SP0601]

Sulfoglycosphingolipids (sulfatides) [SP0602]

Glucuronosphingolipids [SP0603]

Phosphoglycosphingolipids [SP0604]

Other [SP0600]

Basic glycosphingolipids [SP07]

Amphoteric glycosphingolipids [SP08]

Arsenosphingolipids [SP09]

The term “sphingolipid metabolic pathway” refers not only to the compounds and enzymes referenced in FIG. 1, but also to their naturally occurring precursors and metabolites and enzymes involved in the de novo synthesis of such compounds and their precursors.

A “subject” or “patient” refers to an animal in need of treatment that can be effected by methods of the invention. Animals that can be treated in accordance with the invention include vertebrates, with mammals such as bovine, canine, equine, feline, ovine, porcine, and primate (including humans and non-human primates) animals being particularly preferred examples.

A “therapeutic agent” refers to a drug or compound that is intended to provide a therapeutic effect.

A “therapeutically effective amount” (or “effective amount”) refers to an amount of an active ingredient, e.g., an agent according to the invention, sufficient to effect treatment when administered to a subject in need of such treatment. Accordingly, what constitutes a therapeutically effective amount of a composition according to the invention may be readily determined by one of ordinary skill in the art. In the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

As used herein, the terms “therapy” and “therapeutic” encompasses the full spectrum of prevention and/or treatments for a disease, disorder or physical trauma. A “therapeutic” agent of the invention may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments. The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers, and other biologics such as antibodies and antibody variants, receptor decoys, and other protein-based therapeutics.

### SUMMARY OF THE INVENTION

The present invention provides patentable methods that comprise administering to a patient an anti-S1P antibody (or antibody fragment) to reduce the effective concentration of S1P and a modulator of an enzyme of the sphingolipid metabolic pathway. Such methods can be used to treat patients known or suspected to suffer from diseases and disorders correlated with or otherwise characterized by undesired S1P levels or activity. In preferred embodiments, the anti-S1P antibody is LT1009 and the modulator is an SPHK inhibitor. Such methods can be used, for example, to reduce or eliminate an increase in the absolute concentration of S1P as a result of administering to a patient an anti-S1P antibody or anti-S1P antibody fragment.

These and other aspects and embodiments of the invention are discussed in greater detail in the sections that follow. The foregoing and other aspects of the invention will become more apparent from the following detailed description, accompanying drawings, and the claims. Although methods and materials similar or equivalent to those described herein can be used in the practice or testing of the present invention, suitable methods and materials are described below. In addition, the materials, methods, and examples below are illustrative only and not intended to be limiting.

### BRIEF DESCRIPTION OF THE DRAWINGS

This application contains at least one figure executed in color. Copies of this application with color drawing(s) will be provided upon request and payment of the necessary fee. A brief summary of each of the figures is provided below.

FIG. 1: Diagram of the sphingolipid metabolic pathway.

FIG. 2: Two plots showing the plasma level of S1P in patients. The upper plot shows the total, or absolute, concentration of S1P in plasma in cancer patients participating in phase 1 human clinical testing of LT1009 at each of five different dosages (as indicated in the legend in next to the upper plot), whereas the bottom plot shows the effective concentration of S1P in plasma.

### DETAILED DESCRIPTION OF THE INVENTION

This invention is based on the surprising observation that treatment of patients with a humanized monoclonal antibody against S1P leads to dose-dependent increases in the absolute levels of S1P in patient blood, sera, and/or plasma, although the amount of bioavailable, bioactive “free” S1P does not. Because it may desirable to reduce or prevent a dose-dependent increase in absolute S1P levels upon or following administration of an anti-S1P antibody or anti-S1P antibody fragment, however, the instant invention provides methods that allow subsequent increases in S1P levels to be avoided or reduced, as is described in more detail below.

1. Anti-S1P Antibody Compounds.

The present invention concerns methods that involve administering combinations of compositions that contain anti-S1P agents, particularly anti-S1P antibodies and antibody fragments, and compositions that contain modulators of enzymes of the sphingolipid metabolic pathway in order to that reduce the effective concentration of S1P in patients known or suspected to have a disease or disorder correlated with aberrant levels of S1P.

A. Antibody Preparation

Turning first to anti-S1P antibodies and antibody fragments, as those in the art know, antibody molecules (i.e., immunoglobulins) are large glycoprotein molecules with a molecular weight of approximately 150 kDa and are usually composed of two heavy and two light polypeptide chains. Each heavy chain (H) is approximately 50 kDa, whereas each light chain (L) is approximately 25 kDa. Each immunoglobulin molecule usually consists of two heavy chains and two light chains. The two heavy chains are linked to each other by disulfide bonds, the number of which varies between the heavy chains of different immunoglobulin isotypes. Each light chain is linked to a heavy chain by one covalent disulfide bond. In any given naturally occurring antibody molecule, the two heavy chains and the two light chains are identical, harboring two identical antigen-binding sites, and are thus said to be divalent, i.e., having the capacity to bind simultaneously to two identical molecules.

The light chains of antibody molecules from any vertebrate species can be assigned to one of two clearly distinct types, kappa (k) and lambda (l), based on the amino acid sequences of their constant domains. The ratio of the two types of light chain varies from species to species. As a way of example, the average k to l ratio is 20:1 in mice, whereas in humans it is 2:1 and in cattle it is 1:20.

The heavy chains of antibody molecules from any vertebrate species can be assigned to one of five clearly distinct types, called isotypes, based on the amino acid sequences of their constant domains. Some isotypes have several subtypes. The five major classes of immunoglobulin are immunoglobulin M (IgM), immunoglobulin D (IgD), immunoglobulin G (IgG), immunoglobulin A (IgA), and immunoglobulin E (IgE). IgG is the most abundant isotype and has several subclasses (IgG1, 2, 3, and 4 in humans). The Fc fragment and hinge regions differ in antibodies of different isotypes, thus determining their functional properties. However, the overall organization of the domains is similar in all isotypes.

Anti-S1P antibodies suitable for practice in the methods of the invention can be generated by any suitable method. Particularly preferred are monoclonal antibodies, especially those that have been “humanized” or are considered to be fully human antibodies. The invention preferably employs anti-S1P antibodies (or antibody fragments) generated using recombinant techniques. Any suitable expression system can be employed, after which the antibody is purified.

In order to humanize an anti-S1P antibody, a nonhuman anti-S1P antibody is typically generated first. Here, the murine anti-S1P monoclonal antibody LT1002 was generated as described. See, e.g., commonly owned U.S. patent application Ser. Nos. 11/924,890,12/258,337, 12/258,346, 12/258,353, 12/258,355, 12/258,383, 12/690,033, and 12/794,668.

Briefly, an anti-S1P monoclonal antibody can be prepared as follows. First, a derivatized form of S1P is linked to a carrier protein. The resultant immunogen is used to immunize mice. After boosting and establishing plateaued antibody titers, monoclonal antibodies to S1P are generated using the hybridoma method first described by Kohler, et al., Nature, 256:495 (1975), or by other suitable methods, including by recombinant DNA methods (see, e.g., U.S. Pat. No. 4,816,567). Lymphocytes anti-S1P antibody producing mice are fused with myeloma cells to form hybridomas. Culture medium in which hybridoma cells are grown is then assayed for production of monoclonal antibodies directed against S1P. Preferably, the binding specificity of various monoclonal antibody species is determined by immunoprecipitation or by an in vitro binding assay, such as radioimmunoassay (RIA) or enzyme-linked immunoabsorbant assay (ELISA). Binding affinities for various monoclonal antibody species can also be determined.

After hybridoma cells are then identified that produce anti-S1P antibodies of the desired specificity, affinity, and/or activity, clones are subcloned by limiting dilution procedures and grown by standard methods (Goding, Monoclonal Antibodies: Principles and Practice, pp. 59-103 (Academic Press, 1986)). The monoclonal antibodies secreted by the subclones are suitably separated from the culture medium, ascites fluid, or serum by conventional immunoglobulin purification procedures such as, for example, Protein A-Sepharose, hydroxylapatite chromatography, gel electrophoresis, dialysis, or affinity chromatography.

DNA encoding desired monoclonal antibodies can then be readily isolated from antibody-producing cells and sequenced using conventional procedures (e.g., by using oligonucleotide probes that are capable of binding specifically to genes encoding the heavy and light chains of the monoclonal antibodies). Once isolated, the genes encoding the immunoglobulin heavy and light chains can then be cloned into suitable expression vectors, which can then transfected into host cells such as E. coli cells, simian COS cells, Chinese hamster ovary (CHO) cells, or myeloma cells that do not otherwise produce immunoglobulin protein. Recombinant production of anti-S1P monoclonal antibodies is then conducted to generate such quantities of a antibody species as may be desired. The anti-S1P monoclonal antibody LT1002 is a particularly preferred example of such a recombinantly produced anti-S1P antibody.

After obtaining an anti-S1P monoclonal antibody such as LT1002, additional efforts can be undertaken to further optimize the antibody for human administration. General methods for such antibody “humanization” are described in, for example, U.S. Pat. No. 5,861,155, US19960652558, U.S. Pat. No. 6,479,284, US20000660169, U.S. Pat. No. 6,407,213, US19930146206, U.S. Pat. No. 6,639,055, US20000705686, U.S. Pat. No. 6,500,931, US19950435516, U.S. Pat. No. 5,530,101, U.S. Pat. No. 5,585,089, US19950477728, U.S. Pat. No. 5,693,761, US19950474040, U.S. Pat. No. 5,693,762, US19950487200, U.S. Pat. No. 6,180,370, US19950484537, US2003229208, US20030389155, U.S. Pat. No. 5,714,350, US19950372262, U.S. Pat. No. 6,350,861, US19970862871, U.S. Pat. No. 5,777,085, US19950458516, U.S. Pat. No. 5,834,597, US19960656586, U.S. Pat. No. 5,882,644, US19960621751, U.S. Pat. No. 5,932,448, US19910801798, U.S. Pat. No. 6,013,256, US19970934841, U.S. Pat. No. 6,129,914, US19950397411, U.S. Pat. No. 6,210,671, U.S. Pat. No. 6,329,511, US19990450520, US2003166871, US20020078757, U.S. Pat. No. 5,225,539, US19910782717, U.S. Pat. No. 6,548,640, US19950452462, U.S. Pat. No. 5,624,821, and US19950479752. Efforts used to generate various humanized anti-S1P antibodies, including LT1009, are described in commonly owned U.S. patent application Ser. Nos. 11/924,890,12/258,337, 12/258,346, 12/258,353, 12/258,355, 12/258,383, 12/690,033, and 12/794,668.

As an alternative to humanization, human anti-S1P antibodies can also be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits, et al., Proc. Natl. Acad. Sci. USA, 90:2551 (1993); Jakobovits, et al., Nature, 362:255-258 (1993); Bruggermann, et al., Year in Immuno., 7:33 (1993); and U.S. Pat. Nos. 5,591,669, 5,589,369 and 5,545,807. Human antibodies can also be derived from phage-display libraries (Hoogenboom, et al., J. Mol. Biol., 227:381 (1991); Marks, et al., J. Mol. Biol., 222:581-597 (1991); and U.S. Pat. Nos. 5,565,332 and 5,573,905). As discussed above, human antibodies may also be generated by in vitro activated B cells (see, e.g., U.S. Pat. Nos. 5,567,610 and 5,229,275) or by other suitable methods.

In certain embodiments, the anti-S1P antibody is an antibody fragment. Various techniques have been developed for the production of antibody fragments. Traditionally, these fragments were derived via proteolytic digestion of intact antibodies (see, e.g., Morimoto, et al., Journal of Biochemical and Biophysical Methods 24:107-117 (1992); and Brennan, et al., Science 229:81 (1985)). However, these fragments can now be produced directly by recombinant host cells. For example, Fab′-SH fragments can be directly recovered from E. coli and chemically coupled to form F(ab′)₂ fragments (Carter, et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to the skilled practitioner.

In some embodiments, it may be desirable to use multispecific (e.g., bispecific) anti-S1P antibodies having binding specificities for at least two different epitopes. Exemplary bispecific antibodies may bind to two different sphingolipid species. Bispecific antibodies can be prepared as full length antibodies or antibody fragments (e.g., F(ab′)₂ bispecific antibodies).

Antibodies with more than two valencies are also contemplated. For example, trispecific antibodies can be prepared. Tutt et al., J. Immunol. 147:60 (1991). An anti-S1P antibody (or antibody fragment) comprising one or more binding sites per arm or fragment thereof will be referred to herein as “multivalent” antibody. For example a “bivalent” antibody of the invention comprises two binding sites per Fab or fragment thereof whereas a “trivalent” polypeptide of the invention comprises three binding sites per Fab or fragment thereof. In a multivalent polymer of the invention, the two or more binding sites per Fab may be binding to the same or different antigens. For example, the two or more binding sites in a multivalent polypeptide of the invention may be directed against the same antigen, for example against the same parts or epitopes of said antigen or against two or more same or different parts or epitopes of said antigen; and/or may be directed against different antigens; or a combination thereof. Thus, a bivalent polypeptide of the invention for example may comprise two identical binding sites, may comprise a first binding sites directed against a first part or epitope of an antigen and a second binding site directed against the same part or epitope of said antigen or against another part or epitope of said antigen; or may comprise a first binding sites directed against a first part or epitope of an antigen and a second binding site directed against the a different antigen. However, as will be clear from the description hereinabove, the invention is not limited thereto, in the sense that a multivalent polypeptide of the invention may comprise any number of binding sites directed against the same or different antigens.

Other modifications of anti-S1P antibodies can be employed in the instant methods. For example, the invention also pertains to immunoconjugates comprising an anti-S1P antibody (or antibody fragment) conjugated to a cytotoxic agent such as a toxin (e.g., an enzymatically active toxin of bacterial, fungal, plant or animal origin, or fragments thereof), or a radioactive isotope (for example, a radioconjugate). Conjugates are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

It may be desirable to use an antibody fragment, rather than an intact antibody, to increase penetration of target tissues and cells, for example. In this case, it may be desirable to modify the antibody fragment in order to increase its serum half life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis). See, e.g., U.S. Pat. No. 6,096,871.

Covalent modifications of the anti-S1P antibody (or fragment thereof) are also envisioned for use in the present invention. They may be made by chemical synthesis or by enzymatic or chemical cleavage of the antibody, if applicable. Other types of covalent modifications of the antibody (or antibody fragment) can be introduced into the molecule by reacting targeted amino acid residues of the antibody with an organic derivatizing agent that is capable of reacting with selected side chains or the N- or C-terminal residues. Exemplary covalent modifications of polypeptides are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

B. Pharmaceutical Formulations

Therapeutic formulations of an anti-S1P antibody (or antibody fragment) are prepared for storage by mixing the antibody having the desired degree of purity with optional physiologically acceptable carriers, excipients, or stabilizers (see, e.g., Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

The formulations to be used for in vivo administration must be sterile. This is readily accomplished for instance by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

A preferred formulation for systemic administration of the antibodies used in practicing the invention is disclosed in commonly owned U.S. patent application Ser. No. 12/418,597. These and other anti-S1P antibody formulations are useful for a variety of purposes, including the treatment of diseases, disorders, or physical trauma. Pharmaceutical compositions comprising one or more humanized anti-sphingolipid antibodies of the invention may be incorporated into kits and medical devices for such treatment. Medical devices may be used to administer the pharmaceutical compositions of the invention to a patient in need thereof, and according to one embodiment of the invention, kits are provided that include such devices. Such devices and kits may be designed for routine administration, including self-administration, of the pharmaceutical compositions of the invention. Such devices and kits may also be designed for emergency use, for example, in ambulances or emergency rooms, or during surgery, or in activities where injury is possible but where full medical attention may not be immediately forthcoming (for example, hiking and camping, or combat situations).

2. Sphingolipid Metabolic Pathway Inhibitors.

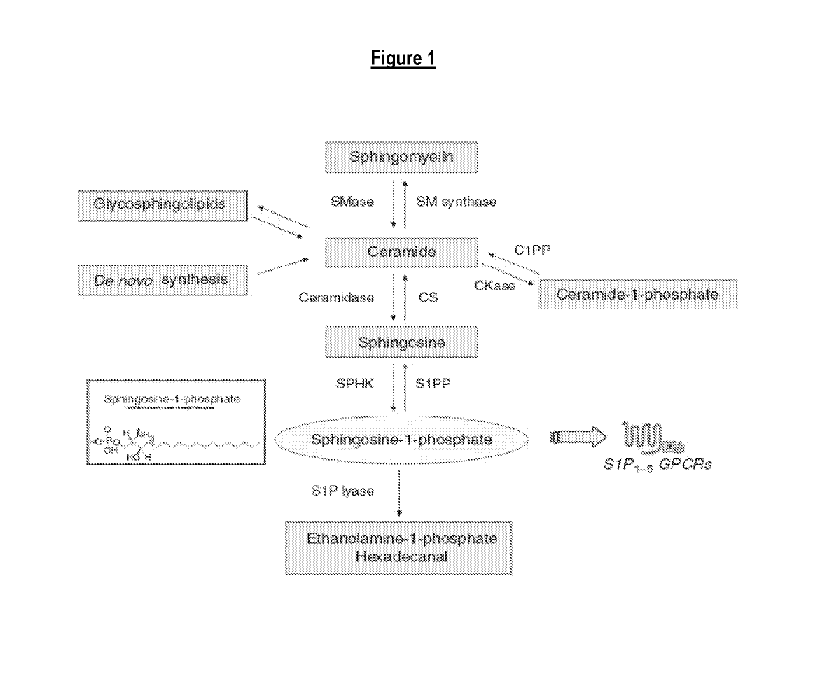

In order to reduce S1P production, inhibitors of one or more enzymes of the sphingolipid metabolic pathway (FIG. 1) are administered in combination with an anti-S1p antibody or antibody fragment. Briefly, de novo sphingolipid synthesis begins with formation of 3-keto-dihydrosphingosine by serine pal mitoyltransferase, which is then reduced to form dihydrosphingosine. Dihydrosphingosine is acylated by a (dihydro)-ceramide synthase (also termed as CerS or CS), to form dihydroceramide. This is desaturated to form ceramide. Ceramide may then be phosphorylated by ceramide kinase to form ceramide-1-phosphate. Alternatively, it may be glycosylated by glucosylceramide synthase or galactosylceramide synthase. Additionally, it can be converted to sphingomyelin by the addition of a phosphorylcholine headgroup by sphingomyelin synthase. Finally, ceramide can serve as the substrate for ceramidase to form sphingosine, which may be phosphorylated to by a sphingosine kinase to form S1P. S1P may be dephosphorylated to reform sphingosine.

Salvage pathways allow the reversion of these metabolites to ceramide. For example, complex glycosphingolipids can be hydrolyzed to glucosylceramide and galactosylceramide, which can then be hydrolyzed by beta-glucosidases and beta-galactosidases to regenerate ceramide. Similarly, sphingomyelin may be broken down by sphingomyelinase to form ceramide. Sphingolipids can be converted to non-sphingolipids through sphingosine-1-phosphate lyase, which catalyzes the formation of ethanolamine phosphate and hexadecenal.

As previously described, sphingosine kinase (SPHK) is an enzyme in the sphingolipid metabolic pathway that catalyzes the phosphorylation of sphingosine to sphingosine-1-phosphate (S1P). Two SPHK isoforms, SPHK 1 and SPHK 2, have been reported. Each exhibits distinct functions. SPHK 1 promotes cell growth and survival. Its expression is up-regulated in cancers, including leukemia, and it has been associated with cancer progression. On the other hand, SPHK 2, when overexpressed, has opposite effects.

SPHK 1 is a key enzyme that regulates the S1P/ceramide rheostat, and S1P and SPHK 1 have long been implicated in resistance of both primary leukemic cells and leukemia cell lines to apoptosis induced by commonly used cytotoxic agents. S1P's precursors, sphingosine and ceramide, are associated with growth arrest and induction of apoptosis, whereas S1P regulates many processes important for cancer progression, including cell growth and survival. Accordingly, the balance between these interconvertible sphingolipid metabolites has been viewed as a cellular rheostat determining cell fate.

Sphingosine kinase inhibitor 2 (SPHK 12; 4-[[4-(4-chlorophenyl)-2-thiazolyl]amino]-phenol; CAS no. 312636-16-1; molecular formula: C₁₅H₁₁ClN₂₀S; molecular weight: 302.8; U.S. patent application Ser. No. 10/462,954, publication no. 20040034075A1) is a potent, selective inhibitor of SPHK 1 with anti-proliferative activity. French, et al. (2003), Cancer Res., vol. 63:5962-5969. SPHK 12 reportedly exhibits non-ATP-competitive inhibition of human recombinant GST-SPHK 1 with an IC₅₀ value of 0.5 μM, with no inhibition against ERK2, PI3-kinase, or PKCα at concentrations up to 60 μM. SPHK I₂ also reportedly inhibits proliferation of several human cancer cell lines (T-24, MCF-7, NCl/ADR, and MCF-7NP) with IC₅₀ values in the low μM range (0.9-4.6 μM). French, et al. supra.

Paugh, et al. ((2008) Blood, vol. 112, no. 4:1382-1391; U.S. patent application Ser. No. 12/387,228, publication no. 20100035959A1) reported the identification of the sphingosine analog (2R,3S,4E)-N-methyl-5-(4′-pentylphenyl)-2-aminopent-4-ene-1,3-diol, designated SK1-I (BML-258), as a potent, water-soluble, isoenzyme-specific inhibitor of SPHK 1 that not only decreases S1P levels but also increases levels of its proapoptotic precursor, ceramide. SK1-1 reportedly does not inhibit SPHK 2, PKC, or numerous other protein kinases. It also has been reported to decrease growth and survival of human leukemia U937 and Jurkat cells, and to enhance apoptosis and cleavage of Bcl-2. Moreover, SK1-1 reportedly potently induces apoptosis in leukemic blasts isolated from patients with acute myelogenous leukemia while sparing of normal peripheral blood mononuclear leukocytes, and markedly reduces growth of AML xenograft tumors. Paugh, et al., supra.

FTY720 (Fingolimod; 2-amino-2[2-(4-octylphenyl)ethyl]propane-1,3-diol hydrochloride; see, e.g., U.S. Pat. No. 6,004,565) is a synthetic sphingosine analogue having immunosuppressant properties. It also reportedly acts as a ceramide synthase inhibitor. In vivo FTY720 is believed to be phosphorylated and exhibit S1P-like effects through several S1P receptors. In human pulmonary artery endothelial cells, FTY720 has been reported to inhibit ceramide synthase 2 and result in decreased cellular levels of dihydroceramides, ceramides, sphingosine, and S1P but increased levels of dihydrosphingosine and dihydrosphingosine 1-phosphate (DHS1P) mediated by SPHK1 activity. Thus, FTY720 can also be used to modulate the intracellular balance of signaling sphingolipids through ceramide synthase (Berdyshev, et al. (2009), J. Biol. Chem., vol. 284:5467-5477), an enzyme involved in the de novo synthesis of ceramide.

Non-isoenzyme-specific inhibitors of SPHKs, such as L-threo-dihydrosphingosine (safingol) and N,N-dimethylsphingosine (DMS), are cytotoxic to leukemia cells.

These and other inhibitors of enzymes of the sphingolipid metabolic pathway can be used in conjunction with anti-S1P antibodies or antibody fragments in practicing the methods of the invention. They will be delivered as pharmaceutical compositions, typically in liquid form, that will include suitable physiologically acceptable carriers, excipients, and/or stabilizers (see, e.g., Remington's Pharmaceutical Sciences 16th edition, Osol, A. Ed. (1980)). The amount of such a composition administered to a particular patient will depend upon many factors, and will left to the discretion of the attending physician in order to achieve a stabilization, and preferably a reduction in, absolute S1P levels in the patient being treated.

3. Applications

Agents that alter the activity or effective concentration of S1P, or its precursors or metabolites, will be useful in the treatment of diseases and disorders correlated with aberrant S1P levels or activity. These agents, including antibodies, act by changing the effective concentration of such undesired bioactive lipids. Lowering the effective concentration of S1P, for example, can be said to “neutralize” S1P or its undesired effects, including downstream effects. Here, S1P will be understood to be “undesired” due to its involvement in a disease process, for example, as a signaling molecule, or because it contributes to disease when present in excess.

Without wishing to be bound by any particular theory, it is believed that inappropriate concentrations of bioactive lipids such as S1P and/or its metabolites or downstream effectors can cause or contribute to the development of various diseases and disorders. As such, the instant methods can be used to treat such diseases and disorders, particularly by decreasing the effective in vivo effective concentration of S1P. In particular, it is believed that the compositions and methods of the invention are useful in treating diseases characterized, at least in part, by aberrant neovascularization, angiogenesis, fibrogenesis, fibrosis, scarring, inflammation, and immune response.

Examples of diseases that may be treated with antibodies targeted to bioactive lipid are described below in applicant's pending patent applications and issued patents. See, for example, commonly owned U.S. patent application Ser. Nos. 11/924,890,12/258,337, 12/258,346, 12/258,353, 12/258,355, 12/258,383 and commonly owned U.S. patent application Ser. Nos. 11/925,173 and 12/446,723.

One way to control the amount of undesirable sphingolipids in a patient is by providing a composition that comprises one or more humanized anti-sphingolipid antibodies to bind one or more sphingolipids, thereby acting as therapeutic “sponges” that reduce the level of free undesirable sphingolipids. When a compound is referred to as “free”, the compound is not in any way restricted from reaching the site or sites where it exerts its undesirable effects. Typically, a free compound is present in blood and tissue, which either is or contains the site(s) of action of the free compound, or from which a compound can freely migrate to its site(s) of action. A free compound may also be available to be acted upon by any enzyme that converts the compound into an undesirable compound.

Without wishing to be bound by any particular theory, it is believed that in certain disease states the amount of S1P (or its metabolites or precursors) rises to undesirable levels, which causes or contributes to the development or progression of the particular disease or disorder, including cardiac and myocardial diseases and disorders.

Because sphingolipids are also involved in fibrogenesis and wound healing of liver tissue, healing of wounded vasculatures, and other disease states or disorders, or events associated with such diseases or disorders, such as cancer, angiogenesis, various ocular diseases associate with excessive fibrosis and inflammation, the compositions and methods of the present disclosure may be applied to treat these diseases and disorders as well as cardiac and myocardial diseases and disorders.

One form of sphingolipid-based therapy involves manipulating the metabolic pathways of sphingolipids in order to decrease the actual, relative, and/or available in vivo concentrations of undesirable, toxic sphingolipids. The invention provides compositions and methods for treating or preventing diseases, disorders or physical trauma, in which humanized anti-sphingolipid antibodies are administered to a patient to bind undesirable, toxic sphingolipids (e.g., S1P), or metabolites thereof.

4. Methods of Administration.

The treatment for diseases and conditions discussed herein can be achieved by administering agents and compositions of the invention by various routes employing different formulations and devices. Suitable pharmaceutically acceptable diluents, carriers, and excipients are well known in the art. One skilled in the art will appreciate that the amounts to be administered for any particular treatment protocol can readily be determined. Suitable amounts of anti-S1P antibodies might be expected to fall within the range of 10 μg/close to 10 g/dose, preferably within 10 mg/dose to 1 g/dose.

Drug substances may be administered by techniques known in the art, including but not limited to systemic, subcutaneous, intradermal, mucosal, including by inhalation, and topical administration. The mucosa refers to the epithelial tissue that lines the internal cavities of the body. For example, the mucosa comprises the alimentary canal, including the mouth, esophagus, stomach, intestines, and anus; the respiratory tract, including the nasal passages, trachea, bronchi, and lungs; and the genitalia. For the purpose of this specification, the mucosa also includes the external surface of the eye, i.e., the cornea and conjunctiva. Local administration (as opposed to systemic administration) may be advantageous because this approach can limit potential systemic side effects, but still allow therapeutic effect.

Pharmaceutical compositions used in the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations used in the present invention may be prepared according to conventional techniques well known in the pharmaceutical industry. Such techniques include the step of bringing into association the active ingredients with the pharmaceutical carrier(s) or excipient(s). Preferred carriers include those that are pharmaceutically acceptable, particularly when the composition is intended for therapeutic use in humans. For non-human therapeutic applications (e.g., in the treatment of companion animals, livestock, fish, or poultry), veterinarily acceptable carriers may be employed. In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment the pharmaceutical compositions may be formulated and used as foams. Pharmaceutical foams include formulations such as, but not limited to, emulsions, microemulsions, creams, jellies, and liposomes.

While basically similar in nature these formulations vary in the components and the consistency of the final product. The know-how on the preparation of such compositions and formulations is generally known to those skilled in the pharmaceutical and formulation arts and may be applied to the formulation of the compositions of the present invention.

In one embodiment, the compositions used in practicing the invention can be delivered to the eye via, for example, topical drops or ointment, periocular injection, intracamerally into the anterior chamber or vitreous, via an implanted depot, or systemically by injection or oral administration. The quantity of antibody and inhibitor used can be readily determined by one skilled in the art.

The traditional approaches to delivering therapeutics to the eye include topical application, redistribution into the eye following systemic administration or direct intraocular/periocular injections [Sultana, et al. (2006), Current Drug Delivery, vol 3: 207-217; Ghate and Edelhauser (2006), Expert Opinion, vol 3: 275-287; and Kaur and Kanwar (2002), Drug Develop Industrial Pharmacy, vol 28: 473-493]. Anti-S1P or other anti-bioactive lipid antibody therapeutics would likely be used with any of these approaches although all have certain perceived advantages and disadvantages. Topical drops are convenient, but wash away primarily because of nasolacrimal drainage often delivering less than 5% of the applied drug into the anterior section of the eye and an even smaller fraction of that dose to the posterior segment of the globe. Besides drops, sprays afford another mode for topical administration. A third mode is ophthalmic ointments or emulsions can be used to prolong the contact time of the formulation with the ocular surface although blurring of vision and matting of the eyelids can be troublesome. Such topical approaches are still preferable, since systemic administration of therapeutics to treat ocular disorders exposes the whole body to the potential toxicity of the drug.

Treatment of the posterior segment of the eye is medically important because age-related macular degeneration, diabetic retinopathy, posterior uveitis, and glaucoma are the leading causes of vision loss in the United States and other developed countries. Myles, et al. (2005), Adv Drug Deliv Rev; 57: 2063-79. The most efficient mode of drug delivery to the posterior segment is intravitreal injection through the pars plana. However, direct injections require a skilled medical practitioner to effect the delivery and can cause treatment-limiting anxiety in many patients. Periocular injections, an approach that includes subconjunctival, retrobulbar, peribulbar and posterior subtenon injections, are somewhat less invasive than intravitreal injections. Repeated and long-term intravitreal injections may cause complications, such as vitreous hemorrhage, retinal detachment, or endophthalmitis.

Pharmaceutical compositions useful in practicing the invention might also be administered using one of the newer ocular delivery systems [Sultana, et al. (2006), Current Drug Delivery, vol 3: 207-217; and Ghate and Edelhauser (2006), Expert Opinion, vol 3: 275-287], including sustained or controlled release systems, such as (a) ocular inserts (soluble, erodible, non-erodible or hydrogel-based), corneal shields, e.g., collagen-based bandage and contact lenses that provide controlled delivery of drug to the eye, (b) in situ gelling systems that provide ease of administration as drops that get converted to gel form in the eye, thereby providing some sustained effect of drug in the eye, (c) vesicular systems such as liposomes, niosomes/discomes, etc., that offers advantages of targeted delivery, bio-compatibility and freedom from blurring of vision, (d) mucoadhesive systems that provide better retention in the eye, (e) prodrugs (f) penetration enhancers, (g) lyophilized carrier systems, (h) particulates, (i) submicron emulsions, (j) iontophoresis, (k) dendrimers, (l) microspheres including bioadhesive microspheres, (m) nanospheres and other nanoparticles, (n) collasomes, and (o) drug delivery systems that combine one or more of the above stated systems to provide an additive, or even synergistic, beneficial effect. Most of these approaches target the anterior segment of the eye and may be beneficial for treating anterior segment disease. However, one or more of these approaches still may be useful affecting bioactive lipid concentrations in the posterior region of the eye because the relatively low molecular weights of the lipids will likely permit considerable movement of the lipid within the eye. In addition, the antibody introduced in the anterior region of the eye may be able to migrate throughout the eye especially if it is manufactured in a lower weight antibody variant such as a Fab fragment. Sustained drug delivery systems for the posterior segment such as those approved or under development could also be employed.

As previously mentioned, the treatment of disease of the posterior retina, choroids, and macula is medically very important. In this regard, transscleral iontophoresis [Eljarrat-Binstock and Domb (2006), Control Release, 110: 479-89] is an important advance and may offer an effective way to deliver antibodies to the posterior segment of the eye.

Various excipients might also be added to the formulated antibody to improve performance of the therapy, make the therapy more convenient or to clearly ensure that the formulated antibody is used only for its intended, approved purpose. Examples of excipients include chemicals to control pH, antimicrobial agents, preservatives to prevent loss of antibody potency, dyes to identify the formulation for ocular use only, solubilizing agents to increase the concentration of antibody in the formulation, penetration enhancers and the use of agents to adjust isotonicity and/or viscosity. Inhibitors of, e.g., proteases, could be added to prolong the half life of the antibody. In one embodiment, the antibody is delivered to the eye by intravitreal injection in a solution comprising phosphate-buffered saline at a suitable pH for the eye.

The anti-S1P agent (e.g., a humanized antibody) and/or sphingolipid pathway inhibitor can also be chemically modified to yield a pro-drug that is administered in one of the formulations or devices previously described above. The active form of the drug is then released by action of an endogenous enzyme. Possible ocular enzymes to be considered in this application are the various cytochrome p450s, aldehyde reductases, ketone reductases, esterases or N-acetyl-β-glucosamidases. Other chemical modifications to the antibody could increase its molecular weight, and as a result, increase the residence time of the antibody in the eye. An example of such a chemical modification is pegylation [Harris and Chess (2003), Nat Rev Drug Discov; 2: 214-21], a process that can be general or specific for a functional group such as disulfide [Shaunak, et al. (2006), Nat Chem Biol; 2:312-3] or a thiol [Doherty, et al. (2005), Bioconjug Chem; 16: 1291-8].

5. Therapeutic Uses

For therapeutic applications, anti-S1P antibodies and sphingolipid pathway inhibitors are administered to a mammal, preferably a human, in a pharmaceutically acceptable dosage form such as those discussed above, including those that may be administered to a human intravenously as a bolus or by continuous infusion over a period of time, by intramuscular, intraperitoneal, intra-cerebrospinal, subcutaneous, intra-articular, intrasynovial, intrathecal, oral, topical, or inhalation routes.

For the prevention or treatment of disease, the appropriate dosages will depend on the type of disease to be treated, as defined above, the severity and course of the disease, whether the antibody is administered for preventive or therapeutic purposes, previous therapy, the patient's clinical history and response to the antibody, and the discretion of the attending physician. The compositions containing antibody and inhibitor are suitably administered to the patient at one time or over a series of treatments.

Depending on the type and severity of the disease, in the context of the anti-S1P antibody, about 1 ug/kg to about 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to about 50 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging.

According to another embodiment of the invention, the effectiveness of the antibody in preventing or treating disease may be improved by administering the antibody serially or in combination with another agent that is effective for those purposes, such as chemotherapeutic anti-cancer drugs, for example. Such other agents may be present in the composition being administered or may be administered separately. The antibody is suitably administered serially or in combination with the other agent.

6. Articles of Manufacture

In another aspect of the invention, articles of manufacture containing materials useful for practicing the instant methods are provided. Such articles comprise one or more containers that contain an anti-S1P antibody composition and a modulator, preferably an inhibitor, of an enzyme in the sphingolipid metabolic pathway, along with a label. Suitable containers include, for example, bottles, vials, syringes, and test tubes. The containers may be formed from a variety of materials such as glass or plastic. The container(s) holds a composition intended to be effective for treating the condition being treated, and may have a sterile access port (for example, the container may be an intravenous solution bag or a vial having a stopper pierceable by a hypodermic injection needle). Preferably, the containers are packaged into a box or other suitable package adapted for safe storage and transport of its contents. The label is placed on the container or package. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The invention will be better understood by reference to the following Examples, which are intended to merely illustrate the best mode now known for practicing the invention. The scope of the invention is not to be considered limited thereto.

### EXAMPLES

### Example 1

### Purification of LT1009 Antibody with Low S1P Carry-Over

Generating highly pure, highly qualified antibodies for pre-clinical or clinical use is of paramount importance for therapeutic drug development. In addition to being free of cellular proteins, DNA and viruses, the antibody preparation should also not contain any of the antigen, so the antibody is fully active and able to bind its target when administered to a patient. Normally, purification and formulation of an antibody removes the antigen, but after purification of the anti-sphingosine-1-phosphate (S1P) monoclonal antibody, LT1009, significant levels of S1P carried over from the antibody production are sometimes observed, particularly when the antibody is produced in a mammalian expression system, as S1P is synthesized by mammalian cells, including Chinese Hamster Ovary (CHO) cells. During production of LT1009, e.g., from the transfected CHO cell line LH1 275 (ATCC Accession No. PTA-8422), intracellular pools of S1P can be released into the media as a result of normal cellular signaling and/or as a consequence of cell rupture after cell death. The LT1009 antibody expressed in the conditioned medium (supernatant) is able to bind to this S1P. As production continues, more S1P may be released and accumulate in the supernatant as a complex with LT1009. While not wishing to be bound by theory, it is believed that the more time the antibody has in contact with the S1P in the medium, the more of that extracellular S1P could be bound to the LT1009 and carried over into the antibody preparation. When produced in CHO cells, LT1009 antibody preparations may contain in excess of 0.5 moles (50 mole percent, mol %) of S1P per mole of antibody. Thus, in order to reduce the amount of S1P carry-over, steps can be taken in both upstream and downstream processing to minimize the amount of S1P in the crude harvest and to promote removal of that S1P during purification.

S1P Quantification Methods:

The S1P concentrations in various preparations of the LT1009 antibody were measured at WindRose Analytica by RP-HPLC-MS-MS method. Mass spectrometry is rapid and sensitive and, if applied properly, can quantify picogram amounts of analyte. The approach taken in this analytical method is to introduce the S1P into an electrospray mass spectrometer source by reversed phase liquid chromatography (RPC). The RPC step separates the S1P from protein, salts, and other contaminants. Following the chromatographic step the S1P is ionized in the source and passed onto an ion trap mass analyzer. All ions except those of the appropriate mass-to-charge ratio (m/z=380) are ejected from the trap. The remaining ions are fragmented in the ion trap and a specific daughter ion (m/z=264) is monitored. The results verify sample identity in three dimensions of analysis: RPC retention time, parent ion m/z of 380, and daughter ion m/z of 264. Additionally, the MS-MS step maximizes signal-to-noise and therefore increases sensitivity significantly. Since no extraction step is required, there is no need for an internal standard. Additionally, the direct injection of sample into the HPLC-MS increases recovery and sensitivity and decreases complexity and analysis time.

For comparison, the concentration of S1P in extracts of selected antibody preparations was determined using a S1P-quantification ELISA. A 4-fold excess of 1:2 chloroform:methanol was added to 1 mg/ml antibody samples to extract the S1P. The aqueous/organic solution was extensively vortexed and sonicated to disrupt antibody-lipid complexes and incubated on ice. After centrifugation, the soluble fraction was evaporated using a speed-vac, and the dried S1P was resuspened in delipidated human serum. The S1P concentration in the resuspended sample was determined by a competitive ELISA using an anti-S1P antibody and a S1P-coating conjugate. The coating conjugate, a covalently linked S1P-BSA, was prepared by coupling a chemically synthesized thiolated S1P with maleimide-activated BSA. For the S1P standard, mono-layer S1P was solubilized in 1% BSA in PBS (137 mM NaCl, 2.68 mM KCl, 10.1 mM Na2HPO4, 1.76 mM KH2PO4; pH 7.4) by sonication to obtain 10 uM S1P (S1P-BSA complex). The S1P-BSA complex solution was further diluted with delipidated human serum to appropriate concentrations (up to 2 uM). Microtiter ELISA plates (Costar, high-binding plate) were coated with S1P-coating material diluted in 0.1 M sodium carbonate buffer (pH 9.5) at 37° C. for 1 hour. Plates were washed with PBS and blocked with PBS/1% BSA/0.1% Tween-20 for 1 hr at room temperature. For the primary incubation, 0.4 ug/mL biotin-labeled anti-S1P antibody, designated amounts of S1P-BSA complex and samples to be tested were added to wells of the ELISA plates. After 1 hour-incubation at room temperature, plates were washed followed by incubation with 100 ul per well of HRP conjugated streptavidin (1:20,000 dilution) for 1 hour at room temperature. After washing, the peroxidase reaction was developed with TMB substrate and stopped by adding 1 M H2SO4. The optical density was measured at 450 nm using a Thermo Multiskan EX.

Upstream Processing to Minimize S1P:

For upstream processing, culturing CHO cells in serum-free medium (Invitrogen, Cat #10743-029) is preferred because serum contains contaminating S1P that could add to that produced by the CHO cells themselves. In addition to use of serum-free medium, harvesting the antibody from the bioreactor prior to extensive cell death will prevent intracellular pools of S1P from release into the medium. Finally, initiating the downstream processing immediately after harvest minimizes the time the LT1009 spends in the presence of S1P in the conditioned medium and the amount of lipid carried over to the final preparation. Despite attempts to minimize S1P levels during upstream processing, significant S1P (e.g., a 0.1-0.2 molar ratio (10-20 mol %) of bound S1P per mol of antibody) often remains in the crude harvest.

Downstream methods have been developed to remove lipids from antibody preparations in order to generate LT1009 material with very low S1P carry-over levels (<0.4 mol % measured by HPLC-MS-MS).

Downstream Processing to Reduce S1P:

Traditionally, purification of antibodies from cultured supernatant or ascites fluid involves affinity chromatography. This one-step method uses recombinant Protein A covalently bound to highly cross-linked agarose (GE healthcare, Cat No 17-5199-04). The Protein A acts as a ligand for Fc domains of monoclonal antibodies. Since the protein-A and S1P binding sites are distinct, S1P does not displace when LT1009 binds the protein-A resin. The high affinity for LT1009 and low solubility in aqueous buffers ensures that S1P remains associated with LT1009 even through extensive washes with high salt buffers (see below). Therefore, a conventional antibody purification process that included: Protein A Chromatography, Low pH Viral Inactivation, followed by Neutralization, Q Anion Exchange Chromatography, Viral Nanofiltration and Final Ultrafiltration/Diafiltration did not remove co-purified (bound to LT1009) S1P. To dissociate S1P from Protein A-bound LT1009, a special feature in the mechanism of binding can be exploited.

Research demonstrated that S1P binding activity of LT1009 was reduced at pH<4.0, or at pH>8.5. However, conducting Protein A chromatography at pH<4.0 in order to reduce bound S1P was not feasible because antibody will not bind to Protein A resin at such low pH. Therefore, a high salt, pH 8.5 wash step was incorporated in Protein A chromatography to reduce S1P bound to LT1009. Further studies demonstrated that the high salt buffer (650 mM NaCl) and 50 mM Sodium Phosphate buffer, pH 8.5 did not effectively remove S1P from LT1009. Further increasing of salt concentration from 0.65 M to 1 M (pH 8.5) and extending of the high salt wash step from four column volumes to five column volumes did not yield product with lower bound S1P.

Use of Metal Chelators to Remove S1P:

A method chelating was developed that involved premixing of two volumes of crude LT1009 antibody harvest, produced from CHO cells bioreactor campaign, with one volume of Protein A IgG binding buffer (“Pierce binding buffer,” Pierce Protein Research Products, Thermo Fisher Scientific, Rockford Ill.), containing 50 mM Potassium Phosphate, 1M NaCl, 2 mM EDTA and 5% glycerol, pH 8.0. According to this procedure the Protein A column was equilibrated with Pierce binding buffer, loaded with premixed crude harvest and washed with 10 column volumes of the same binding buffer. The resulting purified LT1009 contained 2-fold less mole percent of S1P as judged by the S1P-quantification ELISA.

It is currently believed that a metal chelator (e.g., EDTA) is important or even essential for effective reduction of S1P carryover in LT1009 antibody preparations. Indeed, titration of LT1009 with EDTA, which chelates divalent metal cations, abrogates S1P binding. The ability of EDTA to dissociate S1P from LT1009 is believed to facilitate removal of S1P during purification of LT1009. Addition of 2 mM EDTA in the binding and washing buffers effectively lowered the S1P carryover twofold in the eluted antibody fractions. It should be noted that the S1P levels in this study are relatively low initially, and including EDTA should produce greater reduction in lipid carryover in samples with higher initial S1P levels. Without being limited by the following examples, other metal chelators such as EGTA, histidine, malate, and phytochelatin may be useful in dissociating S1P from the antibody. EGTA and EDTA are presently preferred divalent metal chelators for separating S1P from anti-S1P antibodies.

Based on these results, a new high salt buffer was developed by Lpath that was comparable in pH and conductivity to the Pierce binding buffer, and the new premixing step was incorporated in the LT1009 manufacturing process.

### Downstream Purification Process Includes:

Premixing of crude harvest with 4× potassium high salt EDTA buffer (200 mM KPi, 4M NaCl, 8 mM EDTA, 20% glycerol, pH 8.0) in ratio of 2 L crude harvest to 0.182 L KPi high salt-EDTA buffer. This step is intended to disrupt and dissociate S1P from LT1009.
Capture of Crude Harvest-High Salt mix on Protein A column and washing the column with 10 column volumes of High Salt-EDTA buffer to remove S1P.
Elution of LT1009 from Protein A resin at low pH (3.6-3.8).
Low pH hold of Protein A Eluate at pH 3.6-3.8 for a viral inactivation followed by neutralization of the eluate to neutral pH.
Sartobind Q anion exchange chromatography to remove residual host cell proteins and nucleotides, as well as leached Protein A.
Nanofiltration using Virosart CPV nanofilter as an additional step for virus removal. —Final UF/DF filtration for protein concentration and final formulation.

Use of Low pH and C8 Resins to Remove S1P:

In addition to the use of metal chelators such as EDTA during the purification, one can also exploit the hydrophobic nature of S1P to remove the lipid from purified antibody preparations. This method involves a two-step process: 1) dissociation of the lipid from the antibody, and 2) physical separation of the lipid from the aqueous environment. A pH induced Lipid removal (pHiL) treatment can be used as an easy, robust method to promote dissociation from antibody preparations.

Antibodies generally exhibit markedly reduced antigen-binding affinity at low pH. Antibodies generated against phospholipids (e.g. S1P and LPA) fail to bind lipids at pH 3.0-3.5, depending on the specific antibody and the lipid. In determining the correct pH to promote dissociation, a pH titration experiment can be performed to determine the pH that abrogates binding yet maintains an intact IgG, such that binding activity is restored once the pH is increased. In other words the antibody should not be irreversibly inactivated. Once this pH has been determined, the antibody is dialyzed against buffer below the critical pH (e.g. 50 mM sodium acetate, pH 3.0-3.5) at 4° C. Under these conditions, both the lipid and antibody exist as isolated components in solution. The dialyzed solution is passed through a material, such as C8 silica resin (e.g., SepPak cartridges, Waters, Cat no WAT036775), that binds the lipid and facilitates separation of the protein free of lipid. As a consequence, the free lipid irreversibly binds the hydrophobic resin (in the case of C8 silica resin) while the antibody flows through without significant loss (˜90% recovery). Most of the lipid can be removed with one pass through the cartridge, but modest gains in lipid removal can be achieved with an additional pass (Table 1, below).

TABLE 1
Lipid removal using pHiL method

| | | | | Antibody |
|---|---|---|---|---|
| | Mole percent of lipid in sample | | | Recovery |
| | (relative to amount of antibody) | | | % Yield |
| Monoclonal | Before | After | After | (after 1^{st} |
| Antibody | treatment | 1^{st} treatment | 2^{nd} treatment | treatment) |
| Murine | 60% | 6.3% | 0.97% | 88% |
| Anti-S1P | | | | |
| Humanized | 46% | 4.3% | 0.81% | 89% |
| Anti-S1P | | | | |
| Humanized | 14 | 4.5 | 6.0 | 91% |
| Anti-LPA | | | | |

The metal chelation and pHiL methods described above can easily be incorporated into a single purification procedure. EDTA is compatible with most buffers and does not adversely affect antibody stability, solubility, or Protein A binding. During purification, washing the bound IgG with copious amount of EDTA-containing buffer will remove a portion of the S1P from the S1P-LT1009 complex as well as potentially dissociate other metal-dependant antigens-antibody complexes. If the EDTA wash does not sufficiently remove the lipid, the eluate from the Protein A column can be treated using the pHiL method. Elution of bound IgG from Protein A is typically achieved using low pH buffers (pH<3.0). If the anti-lipid antibody elutes from the column at a pH or below the critical pH for lipid binding, the sample can simply be applied to the C8 silica resin to remove the lipid. If necessary, the pH can be easily adjusted prior to applying it to the resin.

### Example 2

### Formulations Containing LT1009

1. Introduction

This example describes experiments to assess the stability of several formulations containing the humanized monoclonal antibody LT1009, which specifically binds S1P. LT1009 is an engineered full-length IgG1k isotype antibody that contains two identical light chains and two identical heavy chains, and has a total molecular weight of about 150 kDa. The complementarity determining regions (CDRs) of the light and heavy chains were derived from a murine monoclonal antibody generated against S1P, and further include a Cys to Ala substitution in one of the CDRs. In LT1009, human framework regions contribute approximately 95% of the total amino acid sequences in the antibody, which binds S1P with high affinity and specificity.

The purpose of the testing described in this example was to develop one or more preferred formulations suitable for systemic administration that are capable of maintaining stability and bioactivity of LT1009 over time. As is known, maintenance of molecular conformation, and hence stability, is dependent at least in part on the molecular environment of the protein and on storage conditions. Preferred formulations should not only stabilize the antibody, but also be tolerated by patients when injected. Accordingly, in this study the various formulations tested included either 11 mg/mL or 42 mg/mL of LT1009, as well as different pH, salt, and nonionic surfactant concentrations. Additionally, three different storage temperatures (5° C., 25° C., and 40° C.) were also examined (representing actual, accelerated, and temperature stress conditions, respectively). Stability was assessed using representative samples taken from the various formulations at five different time points: at study initiation and after two weeks, 1 month, 2 months, and 3 months. At each time point, testing involved visual inspection, syringeability (by pulling through a 30-gauge needle), and size exclusion high performance liquid chromatography (SE-HPLC). Circular dichroism (CD) spectroscopy was also used to assess protein stability since above a certain temperature, proteins undergo denaturation, followed by some degree of aggregate formation. The observed transition is referred to as an apparent denaturation or “melting” temperature (Tₘ) and indicate the relative stability of a protein.

2. Materials and Methods

a. LT1009

The formulation samples (˜0.6 mL each) were generated from an aqueous stock solution containing 42 mg/mL LT1009 in 24 mM sodium phosphate, 148 mM NaCl, pH 6.5. Samples containing 11 mg/mL LT1009 were prepared by diluting a volume of aqueous stock solution to the desired concentration using a 24 mM sodium phosphate, 148 mM NaCl, pH 6.5, solution. To prepare samples having the different pH values, the pH of each concentration of LT1009 (11 mg/mL and 42 mg/mL) was adjusted to 6.0 or 7.0 with 0.1 M HCl or 0.1 M NaOH, respectively, from the original 6.5 value. To prepare samples having different NaCl concentrations, 5 M NaCl was added to the samples to bring the salt concentration to either 300 mM or 450 mM from the original 148 mM. To prepare samples having different concentrations of nonionic surfactant, polysorbate-80 was added to the samples to a final concentration of either 200 ppm or 500 ppm. All samples were aseptically filtered through 0.22 μm PVDF membrane syringe filters into sterile, depyrogenated 10 mL serum vials. The vials were each then sealed with a non-shedding PTFE-lined stopper that was secured in place and protected from contamination with a crimped on cap. Prior to placement into stability chambers, the vials were briefly stored at 2-8° C.; thereafter, they were placed upright in a stability chamber adjusted to one of three specified storage conditions: 40° C. (±2° C.)/75% (±5%) relative humidity (RH); 25° C. (±2° C.)/60% (±5%) RH; or 5° C. (±3° C.)/ambient RH. A summary of the formulation variables tested appears in Table 2, below.

TABLE 2
Formulation Summary

| | | |
|---|---|---|
| Polysorbate 80 | NaCl | pH |

LT1009, 11 mg/mL

| | | |
|---|---|---|
| 0.02% | 148 mM NaCl | 7 |
| Polysorbate | | 6.5 |
| | | 6 |
| | 300 mM NaCl | 7 |
| | | 6.5 |
| | | 6 |
| | 450 mM NaCl | 7 |
| | | 6.5 |
| | | 6 |
| 0.05% | 148 mM NaCl | 7 |
| Polysorbate | | 6.5 |
| | | 6 |
| | 300 mM NaCl | 7 |
| | | 6.5 |
| | | 6 |
| | 450 mM NaCl | 7 |
| | | 6.5 |
| | | 6 |

LT1009, 42 mg/mL

| | | |
|---|---|---|
| 0.02% | 148 mM NaCl | 7 |
| Polysorbate | | 6.5 |
| | | 6 |
| | 300 mM NaCl | 7 |
| | | 6.5 |
| | | 6 |
| | 450 mM NaCl | 7 |
| | | 6.5 |
| | | 6 |
| 0.05% | 148 mM NaCl | 7 |
| Polysorbate | | 6.5 |
| | | 6 |
| | 300 mM NaCl | 7 |
| | | 6.5 |
| | | 6 |
| | 450 mM NaCl | 7 |
| | | 6.5 |
| | | 6 |

b. Taking of Samples

Samples of each formulation were analyzed according to the schedule listed in Table 3, below. One vial was used for each storage condition for all time points. On a date when samples were to be taken, vials were pulled from each stability chamber and 150 μL of each sample were transferred into correspondingly labeled separate vials that were placed on the bench for 1 hour prior to testing. The original vial was immediately placed back into the specified stability chamber after withdrawing the aliquot to be tested.

TABLE 3
Drug Product Formulation Study Stability Matrix

| Storage | Intervals (months) | | | | |
|---|---|---|---|---|---|
| Conditions | T = 0 | 0.5 | 1 | 2 | 3 |

Protein Concentration LT1009, 11 mg/mL

| | | | | | |
|---|---|---|---|---|---|
| 40° C. | x, y | x, y | x | x | x, y |
| 25° C. | | x, y | x | x | x, y |
| 5° C. | | x, y | x | x | x, y |

Protein Concentration LT1009, 42 mg/mL

| | | | | | |
|---|---|---|---|---|---|
| 40° C. | x, y | x, y | x | x | x, y |
| 25° C. | | x, y | x | x | x, y |
| 5° C. | | x, y | x | x | x, y |

x = Appearance, pH, SDS-PAGE, SE-HPLC, UV OD-280, IEF
y = Syringeability (performed by aseptically drawing 200 μL of a sample with a 30-gauge needle connected to a disposable 1-mL syringe)

c. Analytical Procedures

For a given time point, aliquots from each sample were subjected to a series of standard analyses, including visual inspection, syringeability, pH, SDS-PAGE (under both reducing and non-reducing conditions), SE-HPLC, and IEF. Protein concentrations were determined by UV spectroscopy (OD-280). Circular dichroism (CD) studies were also performed.

Circular dichroism spectroscopy was performed separately from the formulation studies. An Aviv 202 CD spectrophotometer was used to perform these analyses. Near UV CD spectra were collected from 400 nm to 250 nm. In this region, the disulfides and aromatic side chains contribute to the CD signals. In the far UV wavelength region (250-190 nm), the spectra are dominated by the peptide backbone. Thermal denaturation curves were generated by monitoring at 205 nm, a wavelength commonly used for b-sheet proteins. Data was collected using 0.1 mg/ml samples with heating from 25° C. to 85° C. Data were collected in 1° C. increments. The total time for such a denaturation scan was between 70 and 90 minutes. The averaging time was 2 seconds.

3. Results and Discussion

For all samples analyzed, visual appearance did not change over time. Likewise, syringeability testing demonstrated that samples could be pulled into a syringe equipped with a 30-gauge needle without difficulty. The results of the various analytical tests were consistent, and SE-HPLC was determined to be an excellent stability-indicating method for LT1009. These results showed that increasing salt concentration reduced both the generation of aggregates and the generation of smaller non-aggregate impurities. It was also found that decreasing pH also reduced aggregate and impurity formation. In addition, it was determined that increasing the polysorbate-80 concentration above 200 ppm did not further stabilize LT1009. The SE-HPLC experiments were performed on samples containing 11 mg/mL LT1009, and comparable results were obtained for samples containing 42 mg/mL LT1009, although lower LT1009 concentrations showed less potential for aggregate formation as compared to the higher concentration, indicating that the antibody appeared to be slightly less stable under all conditions tested at the higher concentration.

From the circular dichroism studies, it was found that LT1009 adopts a well-defined tertiary structure in aqueous solution, with well-ordered environments around both Tyr and Trp residues. It also appeared that at least some of the disulfides in antibody molecules experience some degree of bond strain, although this is not uncommon when both intra- and inter-chain disulfides are present. The secondary structure of LT1009 was found to be unremarkable, and exhibited a far UV CD spectrum consistent with R-sheet structure. The observed transition is referred to as an apparent denaturation or “melting” temperature (Tₘ). Upon heating, LT1009 displayed an apparent Tₘ of approximately 73° C. at pH 7.2. The apparent Tₘ increased to about 77° C. at pH 6.0. These results indicate that a slightly acidic pH could enhance long-term stability of aqueous formulations of LT1009. Addition of NaCl and/or polysorbate-80 also provided additional stabilization.

Together, the data from these experiments indicate that LT1009 is most stable around pH 6 and 450 mM NaCl independent of antibody concentration. Indeed, SE-HPLC testing indicated that increasing the salt concentration to 450 mM and decreasing the pH to 6.0 while maintaining the polysorbate-80 concentration at 200 ppm had a very beneficial effect on the stability of LT1009. Inclusion of polysorbate-80 above 200 ppm had no further mitigating effect against aggregate formation, probably because it was already above its critical micelle concentration at 200 ppm. While not wishing to be bound by any particular theory, the fact that aggregate formation in LT1009 was reduced with increasing salt concentration under the studied conditions could indicate that aggregate formation is at least in part based more on ionic interactions between molecules rather than hydrophobic interactions. The observation that lowering the pH from 7 to 6 also reduces aggregate formation could be explained by reduced hydrophobicity of the amino acid histidine at the lower pH. Finally, the observed increased tendency of aggregate formation at increased LT11009 concentration can simply be explained by the greater chance of molecules hitting each other at the right time at the right place for aggregate formation.

As these experiments show, a preferred aqueous LT1009 formulation is one having 24 mM phosphate, 450 mM NaCl, 200 ppm polysorbate-80, pH 6.1. The relatively high tonicity of this formulation should not pose a problem for systemic applications since the drug product will likely be diluted by injection into IV-bags containing a larger volume of PBS prior to administration to a patient.

### Example 3

### Isolation of Fab Fragments from Anti-S1P Monoclonal Antibodies

Treatment of purified whole IgG preparations with the protease papain separates a Fab fragment consisting of both variable domains and the Ck and Ch1 constant domains from the Fc domain, which contains a pair of Ch2 and Ch3 domains. The Fab fragment retains one entire variable region and, therefore, can be used for therapeutic applications, as well as serve as a useful tool for biochemical characterization of a 1:1 interaction between the antibody and epitope. Furthermore, because it lacks the flexibility and, generally, the glycosylation inherent in native purified whole IgG, Fab fragments are generally excellent platforms for structure studies via single crystal x-ray diffraction.

To prepare Fab fragments of a desired antibody (e.g., an anti-S1P antibody such a sLT1009), purified, intact anti-S1P IgG can be digested with activated papain (incubated 10 mg/ml papain in 5.5 mM cysteine-HCL, 1 mM EDTA, 70 μM 2-mercaptoethanol for 0.5 hours at 37° C.) in digestion buffer (100:1 LT1009:papain in 50 mM sodium phosphate pH 7.2, 2 mM EDTA). After 2 hours at 37° C., the protease reaction is quenched with 50 mM iodoacetamide, dialyzed against 20 mM TRIS pH 9, and loaded onto 2×5 ml HiTrap Q columns. The bound protein is eluted with a linear gradient of 20 mM TRIS pH 8, 0.5 M NaCl and collected in 4 ml fractions. The fractions containing the anti-S1P Fab fragment are pooled and loaded onto a protein A column equilibrated with 20 mM TRIS pH 8. The intact antibody and the Fc fragment bind to the resin, while the Fab fragment is present in the flow through fraction. The Fab fragment can then be concentrated using a centricon-YM30 centrifugal concentrator (Millipore, Cat No 4209), dialyzed against 25 mM HEPES pH 7, and stored at 4° C.

### Example 4

### Analytical Methods

This example details several analytical methods useful in the context of the invention.

a. Quantitative ELISA.

Goat-anti human IgG-Fc antibody (Bethyl, Montgomery Tex., cat no. A80-104A, 1 mg/ml) is diluted 1:100 in carbonate buffer (100 mM NaHCO₃, 33.6 mM Na₂CO₃, pH 9.5). Plates are coated with 100 ul/well of coating solution and incubated at 37° C. for 1 hour. The plates are then washed 4× with TBS-T (50 mM Tris, 0.14 M NaCl, 0.05% Tween-20, pH 8.0) and blocked with 200 μl/well TBS/BSA (50 mM Tris, 0.14 M NaCl, +1% BSA, pH 8.0) for 1 hour at 37° C. Samples and standards are prepared on non-binding plates with enough volume to run in duplicate.

The standard is prepared by diluting human reference serum (Bethyl RS10-110; 4 mg/ml) in TBS-T/BSA (50 mM Tris, 0.14 NaCl, 1% BSA, 0.05% Tween-20, pH 8.0) to the following dilutions: 500 ng/ml, 250 ng/ml, 125 ng/ml, 62.5 ng/ml, 31.25 ng/ml, 15.625 ng/ml, 7.8125 ng/ml, and 0.0 ng/ml. The samples are prepared by making appropriate dilutions in TBS-T/BSA so that the samples OD fall within the range of this standard curve, the most linear range being from 125 ng/ml to 15.625 ng/ml. After washing the plates 4 times with TBS-T, 100 μl of the standard/samples preparation is added to each well and incubated at 37° C. for 1 hour. Next, the plates are washed 4 times with TBS-T and then incubated for 1 hour at 37° C. with 100 ul/well of HRP-goat anti-human IgG antibody (Bethyl A80-104P, 1 mg/ml) diluted 1:150,000 in TBS-T/BSA. The plates are washed 4 additional times with TBS-T and developed using 100 μl/well TMB substrate at 4° C. After 7 minutes, the reaction is stopped by adding 100 μl/well of 1 M H₂SO₄. The OD is measured at 450 nm. Data is analyzed using Graphpad Prizm software.

b. Direct-Binding ELISA.

Microtiter ELISA plates (Costar, Corning Inc., Lowell Mass., Cat No. 3361) are coated overnight with either S1P conjugated to delipidated BSA diluted in 0.1M Carbonate Buffer (pH 9.5) at 37° C. for 1 hour. Plates are washed with PBS (137 mM NaCl, 2.68 mM KCl, 10.1 mM Na₂HPO₄, 1.76 mM KH₂PO₄; pH 7.4) and blocked with PBS/BSA/Tween-20 for 1 hour at room temp or overnight at 4° C. For the primary incubation (1 hour at room temp.), a dilution curve (0.4 μg/mL, 0.2 μg/mL, 0.1 μg/mL, 0.05 μg/mL, 0.0125 μg/mL, and 0 μg/mL) of the antibody is prepared (100 μl/well). Plates are washed and incubated with 100 μl/well of HRP conjugated goat anti-mouse (1:20,000 dilution) (Jackson Immunoresearch, West Grove Pa., Cat No 115-035-003) or HRP conjugated goat anti-human (H+L) diluted 1:50,000 (Jackson, Cat No109-035-003) for 1 hour at room temperature. After washing, the peroxidase is developed with Tetramethylbenzidine substrate (Sigma, cat No T0440) and quenched by addition of 1 M H₂SO₄. The optical density (OD) is measured at 450 nm using a Thermo Multiskan EX. The raw data is transferred to the GraphPad software and the concentration of lipid that produced half maximal effect (EC₅₀) and the maximum binding absorbance (Vmax) is calculated using a 4-parameter nonlinear least squares fit of the saturation binding curves.

c. Lipid Competition Assay.

The ability of various lipids in solution to inhibit direct-S1P binding by a particular antibody (or antibody fragment) species is tested using an ELISA assay format. Microtiter ELISA plates (Costar, Cat No. 3361) are coated with S1P diluted in 0.1 M Carbonate Buffer (pH 9.5) at 37° C. for 1 hour. Plates are washed with PBS (137 mM NaCl, 2.68 mM KCl, 10.1 mM Na₂HPO₄, 1.76 mM KH₂PO₄; pH 7.4) and blocked with PBS/BSA/Tween-20 for 1 hour at room temp or overnight at 4° C. For the primary incubation, 0.4 μg/mL of antibody and designated amounts of lipid are added to wells of the ELISA plates and incubated at room temp for 1 hr. Plates are washed and incubated with 100 p per well of HRP conjugated goat anti-mouse (1:20,000 dilution) (Jackson, cat No 115-035-003) or HRP conjugated goat anti-human (H+L) diluted 1:50,000 (Jackson, cat No109-035-003) for 1 hour at room temperature. After washing, the peroxidase reaction is developed with Tetramethylbenzidine substrate and stopped by adding 1 M H₂SO₄. The optical density (OD) is measured at 450 nm using a Thermo Multiskan EX. The maximum binding absorbance (Vmax) and percent inhibition are calculated by linear regression of the Lineweaver-Burke plots using Excel software.

d. Surface Plasmon Resonance.

All binding data is collected on a ProteOn optical biosensor (BioRad, Hercules Calif.). Thiolated lipids are coupled to a maleimide modified GLC sensor chip (Cat. No 176-5011). First, the GLC chip is activated with an equal mixture of sulfo-NHS/EDC for seven minutes followed by a 7 minute blocking step with ethyldiamine. Next, sulfo-MBS (Pierce Co Rockford, Ill., cat #22312) is passed over the surfaces at a concentration of 0.5 mM in HBS running buffer (10 mM HEPES, 150 mM NaCl, 0.005% tween-20, pH 7.4). The thiolated lipid is diluted into the HBS running buffer to a concentration of 10, 1, and 0.1 μM and injected for 7 minutes producing different lipid density surfaces (˜100, ˜300 and ˜1400 RU). Next, binding data for the WT and mutant antibodies is collected using a 3-fold dilution series starting with 25 nM as the highest concentration. Surfaces are regenerated with a 10 second pulse of 100 mM HCl. All data is collected at 25° C. Controls are processed using a reference surface as well as blank injections. In order to extract binding parameters, the data is globally fit using 1-site and 2-site models.

Through the use of these and other analytical methods it has been determined that LT1009 has a higher binding affinity for S1P (less than 100 μM) than S1P receptors, which have affinities ranging from about 8-50 nM. LT1009 to be highly specific for S1P, as determined by a lack of cross-reactivity against 70 different bioactive lipid species.

### Example 5

### Phase 1 Human Clinical Trial Results for LT1009

This example describes some of the results of a multi-center, open-label, single-arm Phase 1 dose escalation study of Sonepcizumab (LT1009) administered weekly by intravenous infusion as a single therapeutic agent to 30 patients with advanced refractory solid tumors, including renal, colorectal, prostate, breast, melanoma, and salivary gland tumors. The objectives of the study included characterizing the safety, tolerability, and dose-limiting toxicities, if any, of Sonepcizumab. The dosages tested were 1, 3, 10, 17, and 24 mg/kg. Sonepcizumab was administered at days 1, 15, 22, and 29 of cycle 1, and then weekly for all subsequent cycles (1 cycle=4 weeks). The initial Sonepcizumab infusions took place over 90 minutes, and were decreased upon subsequent administrations as tolerated. Pharmacodynamics were assessed by measuring antibody binding performance by serial measurements of S1P from patient samples, by assessing absolute lymphocyte counts over time, and by periodically measuring a series of biomarkers, including VEGF, MMP-2, MMP-9, IL-6, IL-8, and PIGF-1.

Of the 30 patients enrolled in the study, 28 were treated and 21 completed the study. No severe adverse events were observed during the study's course, and no dose reductions were required. Two dose interruptions were necessary as a result of infusion-related reactions at the highest dose (24 mg/kg), which was administered to nine patients, although these reactions improved with prophylactic treatment and/or continued weekly infusions. Other adverse reactions observed in small subsets of patients included diarrhea (3 patients), nausea (3 patients), and anemia (1 patient).

Absolute lymphocyte counts decreased acutely, on average by 48%, between hours 1 and 24 post-treatment in patients receiving the 24 mg/kg dosages. Over 7 days lymphocyte counts recovered, at least in part, in these patients. The 1-24 hour assessment was not made for the lower dosages tested; instead, lymphocyte counts were assessed on an average weekly basis, which revealed a dose-dependent decrease across the dosages tested.

The protein biomarkers VEGF, MMP-2, MMP-9, IL-6, IL-8, and PIGF-1 were measured 7 days after dosing, and no clear difference was seen for any marker at the time points tested.

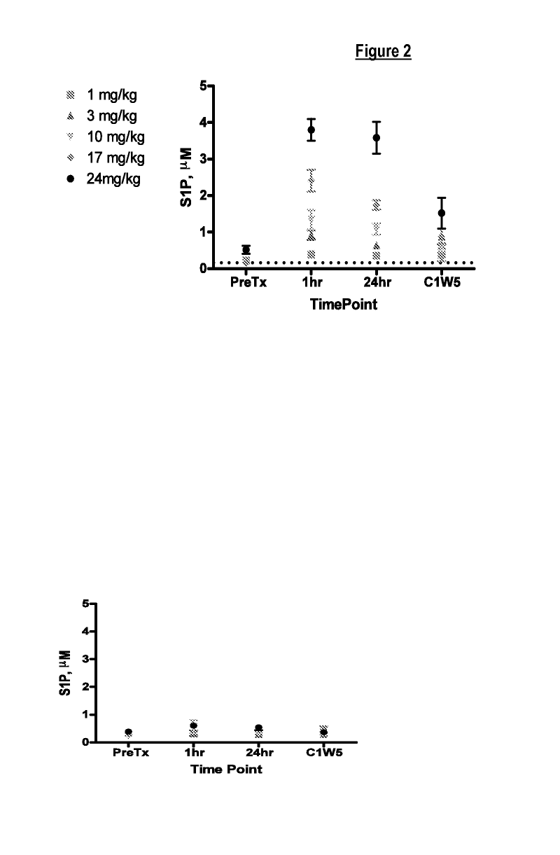

Total, or absolute, S1P exhibited a significant dose-dependent increase (see FIG. 1), although there was no significant does-related change in the amount of bioactive, or “free”, S1P (see FIG. 2). Combination therapy with a modulator of an enzyme of the sphingolipid metabolic pathway could decrease or attenuate the dose-dependent increase in absolute S1P levels.

From this study it was determined that, overall, Sonepcizumab (LT1009) was very well-tolerated, and good exposure was achieved with a weekly dosing schedule. Significantly, evidence of clinical activity was also observed, including one patient with a carcinoid tumor treated with 3 mg/kg/week dosages who has exhibited stable disease since undergoing the initial treatment in September 2008. Stable disease for prolonged periods was also observed in a number of other patients, including 12 months for a patient with adenoid cystic carcinoma and 4-6 months time to progression for patients with melanoma (6 months) and breast, rectal, and renal cancer (4 months).

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference in their entirety for any and all purposes and to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-4. (canceled)

5. A method comprising administering to a patient known or suspected to have a disease or disorder correlated with an aberrant level of S1P a first composition comprising an anti-S 1P antibody or S1P-binding antibody fragment and a second composition comprising a modulator of a sphingolipid metabolic pathway enzyme.

6. A method according to claim 5 that reduces the level of bioavailable S1P in the patient.

7. A method according to claim 5 intended to treat the disease or disorder correlated with an aberrant level of S 1P.

8. A method according to claim 5 wherein the anti-S1P antibody is LT1009 and the modulator is an SPHK inhibitor.
